(19) United States
(12) Patent Application Publication
(10) Pub. No.: US 2017/0086679 A1
(43) Pub. Date: Mar. 30, 2017

(54) PHOTOACOUSTIC APPARATUS AND METHOD FOR ACQUIRING OBJECT INFORMATION

(71) Applicant: CANON KABUSHIKI KAISHA, Tokyo (JP)

(72) Inventors: Takeshi Sekiya, Kawasaki-shi (JP); Kazuhito Oka, Tokyo (JP)

(21) Appl. No.: 15/268,159

(22) Filed: Sep. 16, 2016

(30) Foreign Application Priority Data
Sep. 24, 2015 (JP) 2015-187464

(51) Int. Cl. A61B 5/00; G01N 29/06; G01N 29/24

(52) U.S. Cl. CPC A61B 5/0095; G01N 29/2418; G01N 29/0681; A61B 5/742; A61B 5/748; G01N 2291/02475; G01N 2291/02466; A61B 2576/00

(57) ABSTRACT

A photoacoustic apparatus includes an emitting unit that emits light onto an object; a receiving unit that receives a photoacoustic wave generated from the object irradiated with the light and outputs a signal; a first calculating unit calculates, based on the signal, an initial sound pressure distribution in a first region; a control unit causes the initial sound pressure distribution to be displayed; an acquiring unit acquires first information on a second region, designated within a region of the initial sound pressure distribution, and based on the first information, second information on a third region; a second calculating unit that calculates, based on the first information, propagation of the light in the third region to calculate a light fluence distribution in the second region; and a third calculating unit calculates, based on the initial sound pressure distribution and the light fluence distribution, an object information distribution in the second region.

### BACKGROUND OF THE INVENTION

Field of Invention

The present invention generally relates to a photoacoustic imaging, in particular it relates to an apparatus that acquires object information on the basis of an acoustic wave generated from an object irradiated with pulsed light. A method for acquiring object information, and a non-transitory storage medium storing a program therein are also disclosed.

Description of Related Art

Photoacoustic apparatuses that obtain object information by irradiation of an object with pulsed light are known. Such photoacoustic apparatuses obtain object information by reception of a photoacoustic wave generated from the object owing to the light incident on the object, and analysis of a reception signal of the photoacoustic wave.

Japanese Patent Application Laid-Open No. 2014-140718 discloses a photoacoustic apparatus that displays various types of object information acquired from reception signals of photoacoustic waves. According to Japanese Patent Application Laid-Open No. 2014-140718 the photoacoustic apparatus is capable of switching the display of the various types of object information on the basis of a selection made by a user.

However, when switching and displaying the object information, in some cases, it may take time to provide the object information (an image) to a user because it takes time to acquire and process the object information.

### SUMMARY OF THE INVENTION

Embodiments of the present invention describe a photoacoustic apparatus that can reduce a redundant time that is necessary to provide an image to a user.

A photoacoustic apparatus according to an embodiment includes a light emitting unit that emits light onto an object; a receiving unit that receives a photoacoustic wave generated by irradiation of the object with the light and outputs a signal; a first calculating unit that calculates an initial sound pressure distribution in a first region on the basis of the signal; a control unit that causes the initial sound pressure distribution to be displayed on a display unit; a region acquiring unit that acquires information on a second region designated within a region of the initial sound pressure distribution displayed on the display unit, the second region being smaller than the first region, and information on a third region on the basis of the information on the second region, the third region being larger than the second region and smaller than the first region; a second calculating unit that calculates, on the basis of the information on the third region, propagation of the light emitted onto the object by setting the third region as a calculation region to calculate a light fluence distribution in the second region; and a third calculating unit that calculates, on the basis of the initial sound pressure distribution and the light fluence distribution, an object information distribution in the second region.

Further features of the present invention will become apparent from the following description of exemplary embodiments with reference to the attached drawings.

### BRIEF DESCRIPTION OF THE DRAWINGS

FIG. 1 illustrates a configuration of a photoacoustic apparatus according to a first embodiment.

FIG. 2 illustrates a dataflow according to the first embodiment.

FIG. 3 illustrates a calculation region for a light fluence distribution according to the first embodiment.

FIGS. 4A to 4C illustrate movement of an irradiated region according to the first embodiment.

FIGS. 5A to 5D are flowcharts illustrating an image display method according to the first embodiment.

FIG. 6 illustrates a user interface according to the first embodiment.

FIG. 7 illustrates a dataflow according to a second embodiment.

FIG. 8 is a flowchart illustrating an image display method according to the second embodiment.

FIGS. 9A to 9C are flowcharts illustrating an image display method according to a third embodiment.

FIGS. 10A to 10C illustrate calculation regions for a light fluence distribution according to the third embodiment.

### DESCRIPTION OF THE EMBODIMENTS

A photoacoustic apparatus can image object information related to an optical absorption coefficient of an object by analyzing a photoacoustic wave. An example of the object information acquired by a photoacoustic apparatus according to an embodiment of the present invention is information regarding an optical absorption coefficient or a concentration of a substance constituting an object.

In some cases, a long processing time may be necessary to calculate such object information. A photoacoustic apparatus typically needs a particularly long time to calculate a light fluence distribution.

Accordingly, the photoacoustic apparatus according to an embodiment of the present invention determines a calculation region for a light fluence distribution on the basis of a region of interest designated by a user. In a method for determining the calculation region according to an embodiment of the present invention, it is possible to shorten the time necessary to calculate a light fluence distribution while maintaining or increasing calculation accuracy of a light fluence distribution. The details of the calculation region for the light fluence distribution or the region of interest are described later.

Hereinafter, embodiments of the present invention will be described in detail with reference to the drawings. Note that the same components are basically denoted by the same reference numerals, and repeated description is omitted.

### First Embodiment

A photoacoustic apparatus according to a first embodiment is an apparatus that displays a first optical absorption coefficient distribution corresponding to a first wavelength λ1 by reconstructing a reception signal of a photoacoustic wave obtained by a plurality of irradiations with pulsed light at the first wavelength λ1.

### System Configuration

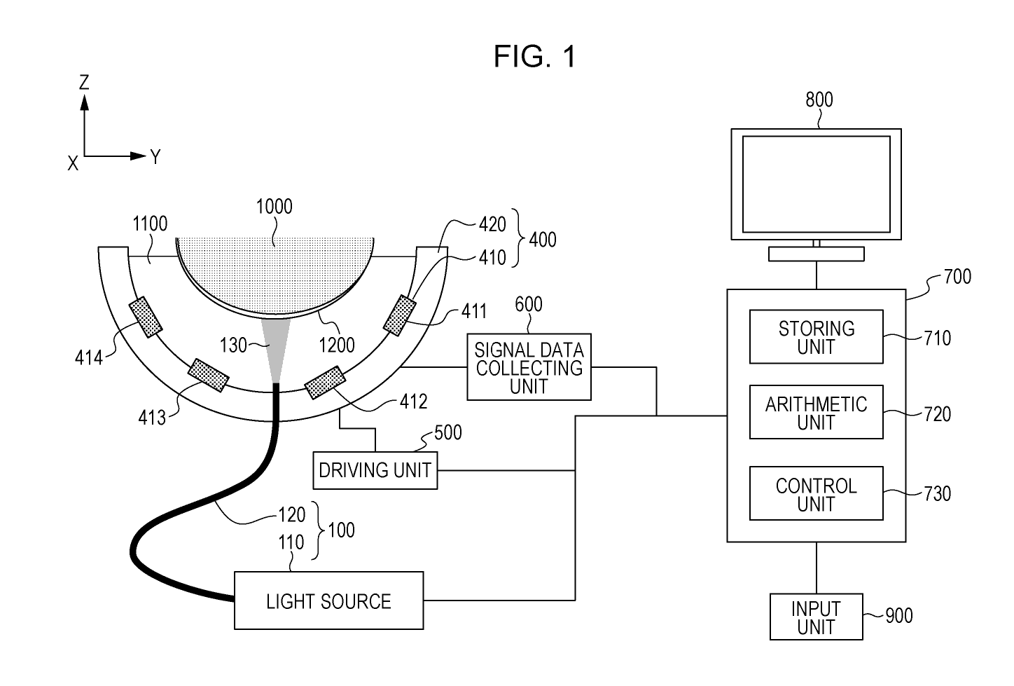

A configuration of the photoacoustic apparatus according to the first embodiment will be described with reference to FIG. 1.

FIG. 1 schematically illustrates the photoacoustic apparatus according to this embodiment. The photoacoustic apparatus includes a light emitting unit 100, a holding unit 1200, a receiving unit 400, a driving unit 500, a signal data collecting unit 600, a computer 700, a display unit 800, and an input unit 900. A measurement target is an object 1000.

### Light Emitting Unit 100

The light emitting unit 100 includes a light source 110 that emits pulsed light 130 and an optical system 120 that guides the pulsed light 130 output from the light source 110 to the object 1000.

The light emitted by the light source 110 may have a pulse width of 1 ns or more and 100 ns or less. The wavelength of the light may be any wavelength in a range of approximately 400 nm to 1600 nm. In the case of imaging a blood vessel just beneath the surface of a living body at a high resolution, light at a wavelength (400 nm or more and 700 nm or less) that is highly absorbed by the blood vessel may be used. On the other hand, in the case of imaging a deep part of a living body, light at a wavelength (700 nm or more and 1100 nm or less) that is not highly absorbed by a background tissue of the living body (e.g., water or fat) may be typically used.

As the light source 110, a laser or a light-emitting diode can be used. If measurement is performed by using light beams at a plurality of wavelengths, a wavelength-changeable light source may be used. If an object is irradiated with light beams at a plurality of wavelengths, a plurality of light sources that generate light beams at mutually different wavelengths can be prepared and the object can be irradiated with the light beams alternately from the respective light sources. In the case of using a plurality of light sources, the light sources are treated as a single light source. As a laser, various lasers, such as a solid-state laser, a gas laser, a dye laser, and a semiconductor laser, can be used. A pulse laser, such as a neodymium-yttrium-aluminum-garnet (Nd:YAG) laser or an alexandrite laser, may be used. In addition, a titanium-sapphire (Ti:Sa) laser or an optical parametric oscillator (OPO) laser, excitation light of which is Nd:YAG laser light, may be used.

As the optical system 120, an optical element, such as a lens, a mirror, or optical fibers, can be used. In the case where the object 1000 is a breast, for example, pulsed light may be emitted with the beam diameter expanded. Accordingly, a light outputting portion of the optical system 120 may be formed of, for example, a diffusion plate that diffuses light. On the other hand, in a photoacoustic microscope, in order to increase the resolution, the light outputting portion of the optical system 120 may be formed of a lens, for example, and may emit light with the beam focused.

Note that the light emitting unit 100 may not include the optical system 120 and may directly irradiate the object 1000 with the pulsed light 130 from the light source 110.

### Holding Unit 1200

The holding unit 1200 is used to hold the object during measurement. Preferably, the holding unit 1200 is configured to accommodate (fit) to the shape of the object. By holding the object 1000 by using the holding unit 1200, the movement of the object 1000 can be suppressed, and the position of the object 1000 can be kept inside the holding unit 1200. The holding unit 1200 can be a receptacle formed of, for example, polyethylene terephthalate glycol-modified (PET-G).

The holding unit 1200 may be made of a material moldable to the shape of the object, but having a hardness high enough to hold the object 1000 in place. The material of the holding unit 1200 may transmit light used for measurement. In addition, the material of the holding unit 1200 may have an acoustic impedance that is substantially equal to that of the object 1000. In the case where the object 1000 has a curved surface, such as the case where the object 1000 is a human breast, the holding unit 1200 may be molded to have a depression. In this case, the object 1000 can be inserted in the depression of the holding unit 1200.

### Receiving Unit 400

The receiving unit 400 includes a receiving element group 410 and a support body 420 that supports the receiving element group 410. The receiving element group 410 consists of receiving elements 411, 412, 413, and 414 that receive an acoustic wave and output electric signals.

Each of the receiving elements 411 to 414 can be formed of a piezoelectric ceramic material typified by lead zirconate titanate (PZT), a polymer piezoelectric film material typified by a polyvinylidene fluoride (PVDF), or the like. An element other than the piezoelectric element may be used. For example, a capacitive micro-machined ultrasonic transducer (CMUT), a transducer using a Fabry-Perot interferometer, or the like can be used. Note that any transducer may be employed as the receiving elements 411 to 414, as long as an electric signal can be output as a result of reception of an acoustic wave.

The support body 420 may be formed of a metal material having a high mechanical strength. In this embodiment, the support body 420 has a hemispherical shell shape and is configured to support the receiving element group 410 on the hemispherical shell. In this case, the directional axes of the respective receiving elements converge at or near the center of curvature of the hemispherical shell. In this manner, the image quality near the center of curvature is increased when imaging is performed by using electric signals output from these receiving elements. Note that the support body 420 may have any configuration as long as the support body 420 can support the receiving element group 410. The plurality of receiving elements 411 to 414 may be arrayed on a plane surface or a curved surface called a 1-dimensional (1D) array, a 1.5D array, a 1.75D array, or a 2D array on the support body 420.

The receiving unit 400 may include an amplifier that amplifies time-sequential analog signals output from the receiving elements. In addition, the receiving unit 400 may include an analog-to-digital (A/D) converter that converts the time-sequential analog signals to time-sequential digital signals, the analog signals having been output from the receiving elements. That is, the receiving unit 400 may include the signal data collecting unit 600.

### Driving Unit 500

The driving unit 500 moves the light emitting unit 100 and the receiving unit 400. The driving unit 500 includes a motor (not shown), such as a stepper motor, which generates a driving force, a driving mechanism that conveys the driving force, and a position sensor (not shown) that detects positional information of the receiving unit 400. As the driving mechanism, a lead screw mechanism, a link mechanism, a gear mechanism, a hydraulic mechanism, or the like can be used. As the position sensor, an encoder, a potentiometer such as a variable resistor, or the like can be used. The driving unit 500 can change the relative position between the object 1000 and the receiving unit 400 linearly one-dimensionally, two-dimensionally, or three-dimensionally. The driving unit 500 can also change the relative position between the object 1000 and the receiving unit 400 rotationally in a circular or elliptical manner (e.g., around the center of curvature of the hemispherical shell). The direction of the axis that intersects both the X direction and the Y direction is herein referred to as a Z direction.

It is sufficient for the driving unit 500 to be capable of changing the relative position between the object 1000 and at least one of the light emitting unit 100 and the receiving unit 400. That is, it is sufficient for the driving unit 500 to move at least one of the object 1000, the light emitting unit 100, and the receiving unit 400 with respect to the other non-moved elements. In the case of moving the object 1000, for example, a configuration in which the object 1000 is moved by movement of the holding unit 1200 that holds the object 1000 may be considered. In addition, the driving unit 500 may move the relative position continuously or in a step-and-repeat manner.

### Signal Data Collecting Unit 600

The signal data collecting unit 600 includes an amplifier circuit that amplifies electric signals, which are analog signals output from the receiving elements 411 to 414, and an A/D converter that converts the analog signals to digital signals, the analog signals having been output from the amplifier. The digital signals output from the signal data collecting unit 600 are stored in a storing unit 710 in the computer 700. The signal data collecting unit 600 is also referred to as a data acquisition system (DAS). The electric signal herein is a concept including both an analog signal and a digital signal. Note that the signal data collecting unit 600 is connected to a light detector attached to the light outputting portion of the light emitting unit 100 and may start a process in synchronization upon output of the pulsed light 130 from the light emitting unit 100 as a trigger.

### Computer 700

The computer 700 includes the storing unit 710, an arithmetic unit 720, and a control unit 730.

The storing unit 710 can be formed of a non-transitory storage medium, such as a magnetic disk or a flash memory. In addition, the storing unit 710 may be a volatile medium, such as a dynamic random access memory (DRAM). Note that a storage medium having a program stored therein is a non-transitory storage medium.

The arithmetic unit 720 can be formed of a processor, such as a central processing unit (CPU) or a graphics processing unit (GPU), or an arithmetic circuit, such as a field programmable gate array (FPGA) chip. These units not only are formed of a single processor or arithmetic circuit, but also may be formed of a plurality of processors or arithmetic circuits.

The control unit 730 is formed of a computing element, such as a CPU. Upon reception of a signal input by any of various operations, such as an image-capturing starting operation, through the input unit 900, the control unit 730 controls each component of the photoacoustic apparatus. In addition, the control unit 730 reads program codes stored in the storing unit 710 and controls operations of each component of the photoacoustic apparatus.

The computer 700 is a device that stores the digital signals output from the signal data collecting unit 600 and that acquires photoacoustic image data of the object 1000 on the basis of the stored digital signals. Processes performed by the computer 700 will be described later in detail.

Note that each function of the computer 700 may be implemented by different pieces of hardware. In addition, the receiving unit 400, the signal data collecting unit 600, and the computer 700 may be implemented by a single piece of hardware. Furthermore, at least some of the components may be implemented by a single piece of hardware. For example, the receiving unit 400 and the signal data collecting unit 600 may be implemented by a single piece of hardware.

### Display Unit 800

The display unit 800 is a liquid crystal display, an organic electroluminescence (EL) display, or another display. The display unit 800 is a device that displays an image, numerals of specific positions, and the like based on optical coefficient information, photoacoustic image data, and the like obtained by the computer 700. The display unit 800 may display an image or a graphical user interface (GUI) for operation of the device.

### Input Unit 900

The input unit 900 can be formed of a mouse, a keyboard, and the like that are operable by a user. In addition, the display unit 800 may be formed of a touch panel so that the display unit 800 can serve as the input unit 900.

Note that each of the components of the photoacoustic apparatus may be formed as an independent device, or the components may be formed integrally as a single device. In addition, at least some of the components of the photoacoustic apparatus may be formed integrally as a single device.

### Object 1000

The object 1000 will be described below although the object 1000 is not a component of the photoacoustic apparatus. The photoacoustic apparatus according to this embodiment may be used for, for example, diagnosis of a malignant tumor, a vascular disease, and the like in a human or an animal, or follow-up of chemotherapy. Accordingly, it is assumed that the object 1000 is a living body, specifically a portion to be diagnosed, such as a breast, a cervix, or an abdomen of a human or an animal. For example, if a human is a measurement target, a light absorber may be oxyhemoglobin, deoxyhemoglobin, a blood vessel containing these in a large amount, a newborn blood vessel formed near a tumor, or the like.

### Acoustic Matching Material 1100

An acoustic matching material 1100 will be described although the acoustic matching material 1100 is not a component of the photoacoustic apparatus. Acoustic waves propagate in the acoustic matching material 1100 between the holding unit 1200 and each of the receiving elements 411 to 414. Water, gel for ultrasonic waves, or the like may be used as the acoustic matching material 1100. The acoustic matching material 1100 may be a material having a small attenuation in acoustic waves. In the case where emitted light passes through the acoustic matching material 1100, the acoustic matching material 1100 may be transparent to the emitted light.

### Dataflow

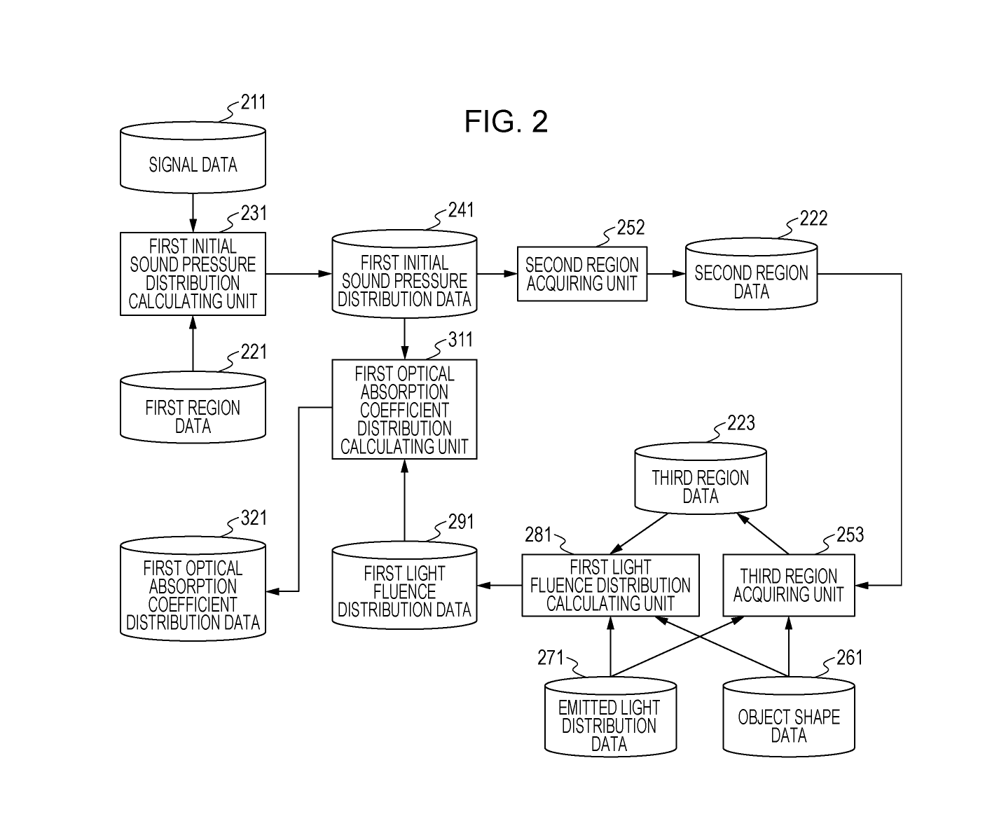

Data processing performed by the computer 700 and data to be handled are described with reference to FIG. 2. Note that each piece of the data is stored in the storing unit 710 and can be input and output.

### Signal Data 211

Signal data 211 is data of an electric signal, which is a first signal, corresponding to the first wavelength λ1, the data being caused by the signal data collecting unit 600 to be stored in the storing unit 710.

### First Region Data 221

First region data 221 is data indicating a two-dimensional or three-dimensional space region. The first region data 221 is data indicating a calculation region for a first initial sound pressure distribution to be calculated by a first initial sound pressure distribution calculating unit 231.

Note that the first region data 221 may be data stored in advance in the storing unit 710. Furthermore, the first region data 221 may be data indicating a two-dimensional or three-dimensional space region designated by a user through the input unit 900. For example, if the region designated by the user has a rectangular shape, a coordinate of a vertex and a coordinate of an opposite vertex correspond to the first region data 221. Note that the shape that can be designated is not limited to a cuboid shape. In addition, the way in which the shape is expressed is not limited to the above example.

### First Initial Sound Pressure Distribution Calculating Unit 231 and First Initial Sound Pressure Distribution Data 241

The first initial sound pressure distribution calculating unit 231 serving as a first calculating unit receives the signal data 211 and the first region data 221, calculates a first initial sound pressure distribution corresponding to the first wavelength λ1 in a first region indicated by the first region data 221, and outputs data of the distribution and as first initial sound pressure distribution data 241.

The first initial sound pressure distribution calculating unit 231 may calculate the first initial sound pressure distribution by using a known reconstruction algorithm, such as a time-domain reconstruction method, a Fourier-domain reconstruction method, or a model-base method (repeated reconstruction method).

Here, an example of employing, as a reconstruction method, universal back-projection (UBP) described in PHYSICAL REVIEW E 71. 016706 (2005) will be described.

In the case of UBP, the first initial sound pressure distribution calculating unit 231 performs preprocessing on the signal data 211 according to the following Expression 1:

$$b ( {r}_{0} , \overline{t} ) = 2 p ( {r}_{0} , \overline{t} ) - 2 \overline{t} \frac{\partial p ( {r}_{0} , \overline{t} )}{\partial t} \qquad Expression 1$$

where p(r₀,t) is a value of a signal acquired by a receiving element at a position r₀ at a time t. In addition, b(r₀,t) is a value obtained as a result of preprocessing on p(r₀,t).

Then, the first initial sound pressure distribution calculating unit 231 splits the region designated according to the first region data 221 into voxels having a predetermined size and calculates an initial sound pressure in each of the voxels. In the case of UBP, the first initial sound pressure distribution calculating unit 231 calculates the initial sound pressure according to the following Expression 2:

$${p}_{0}^{( b )} ( r ) = \frac{{\sum }_{i = 1}^{N} Δ {Ω}_{i} \times b ( {d}_{i} , \overline{t} = \vert {d}_{i} - r \vert )}{{\sum }_{i = 1}^{N} Δ {Ω}_{i}} \qquad Expression 2$$

where p^{(b)}₀(r) is the initial sound pressure in a voxel to be reconstructed, N is the number of receiving elements, and ΔΩᵢ is the weight based on a solid angle subtending an i-th receiving element from the voxel to be reconstructed, the voxel being expressed by the following Expression 3:

$$Δ {Ω}_{i} = \frac{Δ {S}_{i}}{{\vert r - {d}_{i} \vert}^{2}} \cdot [ {n}_{{0}_{i}}^{s} \cdot \frac{( r - {d}_{i} )}{\vert r - {d}_{i} \vert} ] \qquad Expression 3$$

where dᵢ is the position vector of the i-th receiving element, ΔSᵢ is the area of the i-th receiving element, and n⁸₀ᵢ is a unit normal vector with respect to a face of the i-th receiving element in dᵢ.

Then, the first initial sound pressure distribution calculating unit 231 outputs data of the calculated initial sound pressures in the respective voxels as the first initial sound pressure distribution data 241.

### Second Region Acquiring Unit 252 and Second Region Data 222

A second region acquiring unit 252 receives the first initial sound pressure distribution data 241 and outputs second region data 222.

The second region data 222 is data indicating a second region that has been designated by the user through the input unit 900 within the first region and that is smaller than the first region. The second region acquiring unit 252 receives the data indicating a region, the data being output from the input unit 900, and outputs the data as the second region data 222. That is, the input unit 900 is configured in such a manner that the second region smaller than the first region can be input. The second region is a region that indicates a display region of a first optical absorption coefficient and that is desired by the user.

For example, on the image of the first initial sound pressure distribution in the first region, the image being displayed on the display unit 800, the user may designate an arbitrary region through the input unit 900 as the second region.

### Object Shape Data 261

Object shape data 261 is data indicating the shape of the object 1000. In the case where the object 1000 is pressed against the holding unit 1200, the shape of the holding unit 1200 may be set as the shape of the object 1000.

In addition, the object shape may be estimated by using an image obtained by taking a picture of the object 1000 with a camera or another modality.

### Emitted Light Distribution Data 271

Emitted light distribution data 271 is data indicating a light fluence distribution of the pulsed light 130 on the surface of the object 1000. Prior to placing the object 1000 on the photoacoustic apparatus, a light fluence distribution of light emitted onto the surface of a target simulating the object may be measured in advance as a light fluence distribution indicated by the emitted light distribution data 271. In the case where the shape of the target differs from the shape of the object, the emitted light distribution data 271 may be estimated by converting the shape according to the measured data as necessary on the basis of the object shape data 261.

### Third Region Acquiring Unit 253 and Third Region Data 223

A third region acquiring unit 253 receives the second region data 222, the object shape data 261, and the emitted light distribution data 271 and outputs third region data 223.

The third region data 223 is data indicating a third region that includes at least the second region and an irradiated region of the object surface irradiated with the pulsed light 130. In addition, the third region data 223 is data indicating a region that is larger than the second region and smaller than the first region. The third region data 223 will be described later in detail.

On the basis of the object shape data 261 and the emitted light distribution data 271, the third region acquiring unit 253 calculates the irradiated region of the object surface irradiated with the pulsed light 130. Note that the data indicating the irradiated region of the object surface irradiated with the pulsed light 130 may be stored in advance in the storing unit 710. In this case, the third region acquiring unit 253 may read the data indicating the irradiated region of the object surface irradiated with the pulsed light 130, the data being stored in the storing unit 710.

### First Light Fluence Distribution Calculating Unit 281 and First Light Fluence Distribution Data 291

A first light fluence distribution calculating unit 281 serving as a second calculating unit receives the third region data 223, the object shape data 261, and the emitted light distribution data 271, and calculates propagation of light at the first wavelength λ1 emitted onto the object 1000 by setting the third region as a calculation region. As a result, the first light fluence distribution calculating unit 281 acquires a first light fluence distribution corresponding to the first wavelength λ1 in the third region, and outputs data of the first light fluence distribution as first light fluence distribution data 291. Since the third region contains the second region, by setting the third region as a calculation region, the second region is also included in the calculation target. That is, the first light fluence distribution calculating unit 281 calculates a light fluence distribution in the second region as a result of calculation of a light fluence distribution in the third region.

The first light fluence distribution calculating unit 281 uses the object shape data 261 and the emitted light distribution data 271, for example, as boundary conditions. Further, the first light fluence distribution calculating unit 281 can calculate the first light fluence distribution by a method for numerically solving a transport equation or diffusion equation indicating the behavior of light energy in a medium that absorbs and scatters light. As the numerically solving method, a finite element method, a differentiation method, a Monte Carlo method, or the like can be employed. Note that for ease of description, the following description is made by assuming that cubic voxels are split although cubic voxels are not necessarily split depending on the method.

Then, the first light fluence distribution calculating unit 281 outputs data of the calculated light fluence distributions in the respective voxels as the first light fluence distribution data 291.

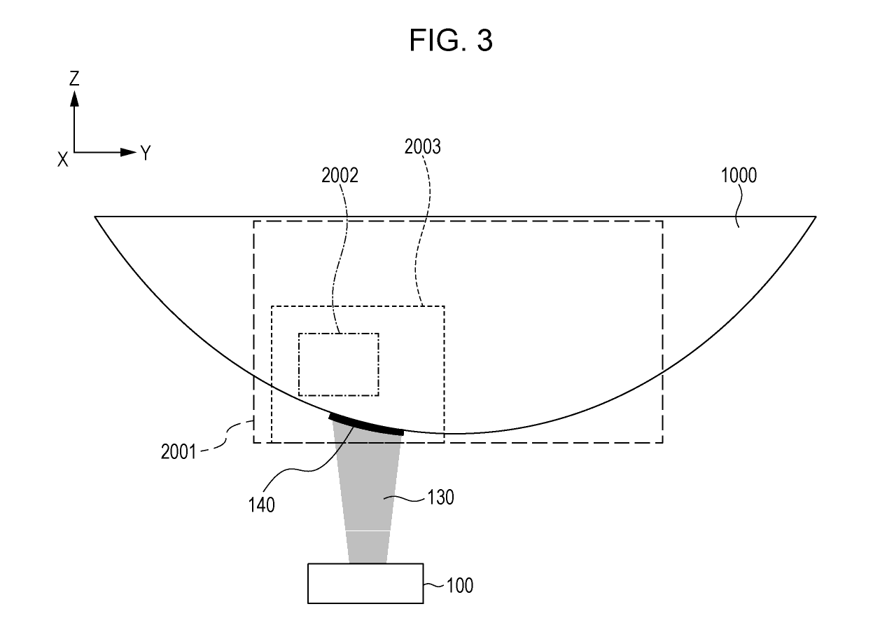

Here, the third region, which is the calculation region of the first light fluence distribution calculating unit 281, will be described with reference to FIG. 3.

The first initial sound pressure distribution calculating unit 231 acquires the first initial sound pressure distribution in a first region 2001. In addition, the second region acquiring unit 252 sets a second region 2002, which is a region of interest desired by the user, within the first region 2001.

The first light fluence distribution calculating unit 281 is to calculate the light fluence distribution corresponding to the first wavelength λ1 in the second region 2002. However, the first light fluence distribution calculating unit 281 needs to calculate the light fluence distribution in the second region 2002 by calculating light propagation from an irradiated region 140 where the object surface is irradiated with the pulsed light 130 to the second region 2002. Therefore, light propagating regions other than the second region 2002 need to be included in a calculation region (third region) 2003 of the first light fluence distribution calculating unit 281.

Furthermore, since light scatters within the object 1000 in various directions, light propagates to the second region 2002 from regions other than the region between the second region 2002 and the irradiated region 140 where the object surface is irradiated with the pulsed light 130. Accordingly, peripheral regions of the second region 2002 may be included in the calculation region (third region) 2003 of the first light fluence distribution calculating unit 281. However, light scattered in regions away from the second region 2002 is not likely to reach the second region 2002. Therefore, the calculation region (third region) 2003 of the first light fluence distribution calculating unit 281 is set as a region larger than the second region 2002 and smaller than the first region 2001.

Then, the first light fluence distribution calculating unit 281 calculates light propagation from the irradiated region 140 to the second region 2002 by setting, as a calculation region, the third region 2003 including the second region 2002, thereby acquiring the light fluence distribution in the second region 2002, and outputs data of the light fluence distribution as the first light fluence distribution data 291.

By calculating the light fluence distribution in the calculation region that has been determined in the above manner, it is possible to shorten a time necessary to calculate the light fluence distribution while suppressing a decrease in calculation accuracy of the light fluence distribution.

As described above, in regions that are sufficiently away from the irradiated region 140, since the light fluence entering these regions is low, the output light fluence by being diffused therein is also low, and such light fluence does not greatly affect other regions. Therefore, even if such regions are omitted from the calculation region, the error is small compared with the case where the calculation region is the entire region of the object. Accordingly, such regions may be omitted from the calculation region.

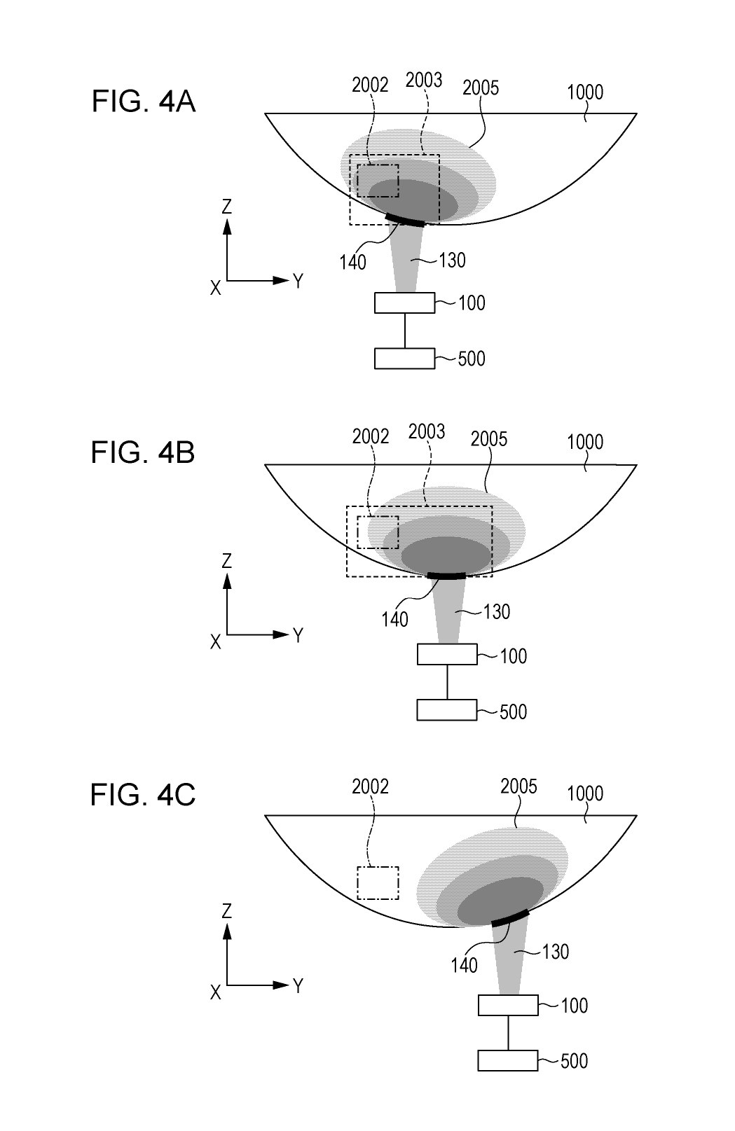

Here, the light fluence distribution calculation region that is set in the case where the driving unit 500 moves a position irradiated with the pulsed light 130, i.e., changes the relative position between the light emitting unit 100 and the object 1000, will be described. FIGS. 4A and 4B illustrate the third region 2003 with respect to the irradiated region 140 at each movement position when the driving unit 500 moves the light emitting unit 100.

First, the third region acquiring unit 253 acquires a light fluence distribution 2005 of the pulsed light 130 in the object 1000, the pulsed light 130 being emitted onto the irradiated region 140 at each movement position, by reading data thereof from the storing unit 710 storing the data in advance or by analytically solving a diffusion equation. Any of these methods enables the light fluence distribution to be acquired more easily than in the case of numerically solving a transport equation or diffusion equation by using the first light fluence distribution calculating unit 281. Note that the storing unit 710 may store in advance light fluence distribution data associated with parameters that are necessary to calculate the light fluence distribution, such as an optical coefficient of the object 1000. Then, the third region acquiring unit 253 may receive the parameters necessary to calculate the light fluence distribution and may read the light fluence distribution data associated with the parameters input from the storing unit 710. FIGS. 4A to 4C each schematically illustrate the light fluence distribution 2005 in a vertical section of the object 1000. In the light fluence distribution 2005 illustrated in FIGS. 4A to 4C, the level of light fluence within the object 1000 is expressed by the color density. The lightest grey portion in the light fluence distribution 2005 represents the minimum light fluence to be included in the calculation region. That is, the light fluence distribution 2005 represents the outer edge of the minimum light fluence to be included in the calculation region. Note that the value of the minimum light fluence to be included in the calculation region can be changed by using the input unit 900, for example.

Then, the third region acquiring unit 253 determines whether or not the second region 2002 and the light fluence distribution 2005 have a common region. If the second region 2002 and the light fluence distribution 2005 have a common region, the third region acquiring unit 253 determines the third region 2003 in accordance with the above-described rule. On the other hand, if the second region 2002 and the light fluence distribution 2005 do not have a common region, the third region acquiring unit 253 determines an empty region to be the third region 2003. That is, if the second region 2002 and the light fluence distribution 2005 do not have a common region, the third region acquiring unit 253 outputs data indicating that the first light fluence distribution calculating unit 281 does not calculate light propagation.

In FIG. 4A, since the second region 2002 and the light fluence distribution 2005 have a common region, the first light fluence distribution calculating unit 281 sets the third region 2003. Similarly, in FIG. 4B, since the second region 2002 and the light fluence distribution 2005 have a common region, the first light fluence distribution calculating unit 281 sets the third region 2003. In contrast, in FIG. 4C, the second region 2002 and the light fluence distribution 2005 do not have a common region. Accordingly, the third region acquiring unit 253 outputs data indicating that the propagation of the pulsed light 130 used for irradiation in this case is not to be calculated.

Here, a result of simulation performed to reveal a change in errors included in the light fluence distribution in the second region depending on the size of the third region will be described.

The simulation has been performed by using the following setting parameters. The object is assumed to be a semi-infinite medium having a homogeneous optical coefficient, the absorption coefficient is set to 0.00534/mm, and the scattering coefficient is set to 0.969/mm. Here, a typical absorption coefficient and a typical scattering coefficient of a living body are set.

As incident light on an X-Y plane of the object, source light according to a Gaussian function with an incident intensity of 100 mJ and a beam diameter of 30 mm has been set.

As the calculation regions (X×Y×Z) for a light fluence distribution, the following five regions have been set: <1>60 mm×60 mm×60 mm, <2> 80 mm×80 mm×60 mm, <3> 100 mm×100 mm×60 mm, <4> 60 mm×60 mm×80 mm, and <5> 120 mm×120 mm×120 mm.

Note that the second region in which the light fluence distribution is to be acquired is assumed to be a region of 60 mm×60 mm×60 mm. That is, the calculation region <1> does not have a margin from the second region, and the second region is identical to the third region. The calculation region <2> is a region having a margin of 10 mm in the X-Y direction from the second region, and the calculation region <3> is a region having a margin of 20 mm in the X-Y direction from the second region. The calculation region <4> is a region having a margin of 10 mm in the Z direction from the second region. By setting the light fluence distribution acquired in the calculation region <5> as the true value of the light fluence distribution in the second region, calculation results for the second region obtained when the calculation regions <1> to <4> are set have been estimated.

As a result of the above simulation, in the light fluence distribution in the second region obtained for the calculation region <1>, a 84% error at maximum has been generated. In the light fluence distribution in the second region obtained for the calculation region <2>, a 18% error at maximum has been generated. In the light fluence distribution in the second region obtained for the calculation region <3>, a 3% error at maximum has been generated. In the light fluence distribution in the second region obtained for the calculation region <4>, a 0.5% error at maximum has been generated.

From these results, it is found that the error decreases as the margin from the second region is increased. It is also found that the error is smaller in the in-plane direction of the irradiated region 140 (e.g., horizontal direction in FIG. 4B) than in the out-of-plane direction (e.g., vertical direction in FIG. 4B) even if the margin from the second region is small. That is, in order to reduce the calculation amount, the margin of the third region from the second region may be smaller in the out-of-plane direction of the irradiated region 140 than in the in-plane direction thereof. In addition, the margin of the third region from the second region may be set only in the in-plane direction, not in the out-of-plane direction. Furthermore, the third region may be set outside the second region by 10 mm or more. Alternatively, the third region may be set outside the second region by 20 mm or more. In addition, the third region may be set outside the second region by 20 mm or more in the in-plane direction of the irradiated region, and the third region may be set outside the second region by 10 mm or more in the out-of-plane direction of the irradiated region.

Note that the user may input a numeral for the margin of the third region from the second region through the input unit 900. For example, the input unit 900 may be configured in such a manner that a numeral for the margin, such as 10 mm or 20 mm, can be directly input. In addition, the input unit 900 may be configured in such a manner that a speed-preference mode and a quantitativeness-preference mode can be selected. The third region acquiring unit 253 may set the third region corresponding to the mode obtained from the input unit 900.

### First Optical Absorption Coefficient Distribution Calculating Unit 311 and First Optical Absorption Coefficient Distribution Data 321

A first optical absorption coefficient distribution calculating unit 311 serving as a third calculating unit receives the second region data 222, the first initial sound pressure distribution data 241, and the first light fluence distribution data 291. Then, the first optical absorption coefficient distribution calculating unit 311 calculates a first optical absorption coefficient distribution corresponding to the first wavelength λ1 in the second region 2002 and outputs data of the distribution as first optical absorption coefficient distribution data 321. That is, the first optical absorption coefficient distribution calculating unit 311 serving as an object information calculating unit calculates the first optical absorption coefficient distribution as an object information distribution.

The following Expression 4 represents an initial sound pressure P₀ obtained when a photoacoustic wave is generated:

P₀(r)=Γ(r)·μₐ(r)·Φ(r) Expression 4

where r represents a position within the object. P₀ represents an initial sound pressure and is obtained on the basis of a reception signal of a photoacoustic wave. In addition, Γ represents a Grueneisen constant, which is a known parameter uniquely determined upon determination of a tissue, μₐ, represents an optical absorption coefficient, and Φ represents a light fluence.

The first optical absorption coefficient distribution calculating unit 311 can acquire the optical absorption coefficient μₐ by dividing the initial sound pressure P₀ at each position in the second region 2002 by the light fluence Φ according to Expression 4. Note that the Grueneisen constant Γ is known, and the first optical absorption coefficient distribution calculating unit 311 may use the Grueneisen constant for calculation by reading data thereof stored in advance in the storing unit 710.

The first optical absorption coefficient distribution calculating unit 311 splits the second region 2002 corresponding to the second region data 222 into voxels having a predetermined size. The first optical absorption coefficient distribution calculating unit 311 calculates the optical absorption coefficient according to Expression 4 for each of the voxels in the second region 2002. Here, in some cases, the voxel size according to the first initial sound pressure distribution data 241, the voxel size according to the first light fluence distribution data 291, and the voxel size used in splitting by the first optical absorption coefficient distribution calculating unit 311 may be different from one another. In this case, the first optical absorption coefficient distribution calculating unit 311 needs to perform resampling on the voxel values of the first initial sound pressure distribution data 241 and the first light fluence distribution data 291 to the voxel used for calculation of the optical absorption coefficient distribution. This can be performed by using a known technique such as nearest neighbor interpolation or linear interpolation.

The first initial sound pressure distribution data 241 is data indicating the initial sound pressure distribution in the first region 2001 including the second region 2002, and the first light fluence distribution data 291 is data indicating the first light fluence distribution in the third region 2003 including the second region 2002. Accordingly, the first optical absorption coefficient distribution calculating unit 311 can acquire the first optical absorption coefficient distribution in the second region 2002 by using the first initial sound pressure distribution data 241 and the first light fluence distribution data 291.

Then, the first optical absorption coefficient distribution calculating unit 311 outputs data of the calculated optical absorption coefficients in the respective voxels as the first optical absorption coefficient distribution data 321.

### Image Display Method

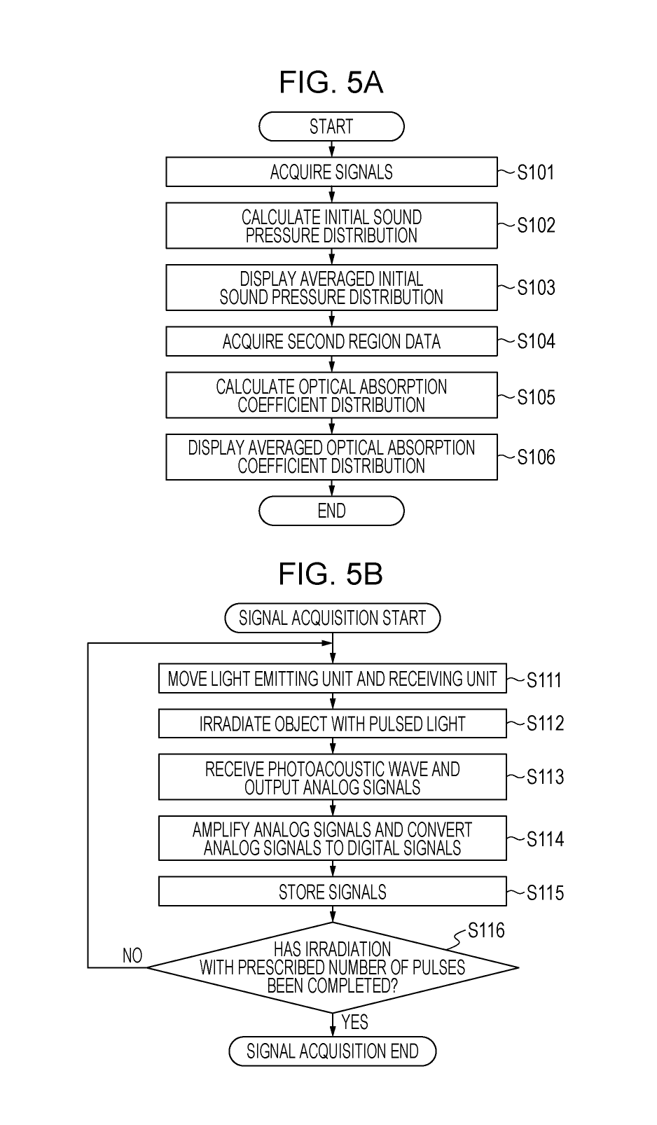

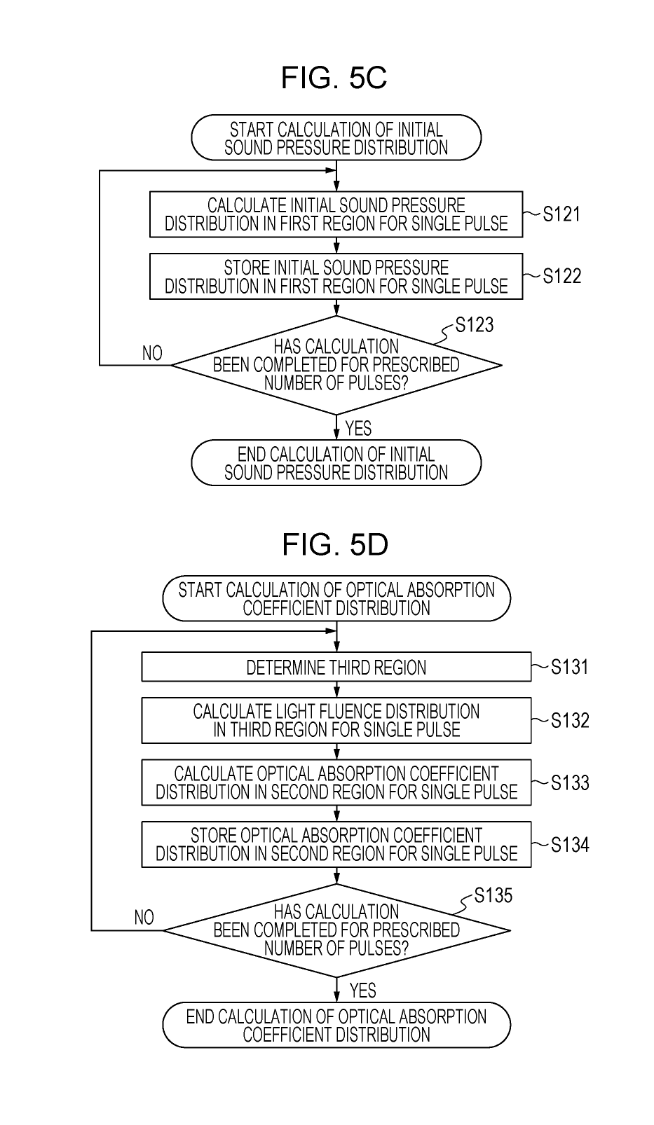

A method for displaying an image of the optical absorption coefficient distribution by using the photoacoustic apparatus according to this embodiment will be described with reference to FIGS. 5A to 5D. FIG. 5A is a flowchart illustrating an overall method for acquiring object information, and FIGS. 5B, 5C, and 5D are flowcharts respectively illustrating details of signal acquisition, initial sound pressure distribution calculation, and optical absorption coefficient distribution calculation.

First, signals are acquired (S101).

Here, the signal acquisition will be described in detail with reference to FIG. 5B. The driving unit 500 moves the light emitting unit 100 and the receiving unit 400 to desired positions (S111). Then, the light emitting unit 100 irradiates the object 1000 with the pulsed light 130 (S112). Then, the pulsed light 130 is absorbed by the object 1000, and a photoacoustic wave is generated by the photoacoustic effect. The receiving unit 400 receives the photoacoustic wave and outputs electric signals that are time-sequential analog signals (S113). The signal data collecting unit 600 collects the time-sequential analog signals output from the receiving unit 400 and performs processes for amplifying the signals and converting the analog signals into digital signals (S114). Then, the signal data collecting unit 600 causes the electric signals, which are time-sequential digital signals, to be stored in the storing unit 710 (S115). Here, the control unit 730 determines whether or not irradiation with a prescribed number of pulses has been completed (S116). If the irradiation has not been completed, the process returns to step S111; if the irradiation has been completed, the signal acquisition ends.

Then, an initial sound pressure distribution is calculated (S102).

Here, the calculation of the initial sound pressure distribution will be described in detail with reference to FIG. 5C. The first initial sound pressure distribution calculating unit 231 calculates a first initial sound pressure distribution for a single pulse (S121) and causes the first initial sound pressure distribution data 241 for a single pulse to be stored in the storing unit 710 (S122). Here, the control unit 730 determines whether or not calculation has been completed for the pulses used for irradiation in step S101 (S123). If the calculation has not been completed, the process returns to step S121; if the calculation has been completed, the calculation of the initial sound pressure distribution ends. Note that since an image to be used to allow the user to select a second region is calculated, a region including the entire object may be set as a first region indicated by the first region data 221 at this time.

Then, initial sound pressure distributions for a plurality of pulses are averaged, and the averaged initial sound pressure distribution is displayed on the display unit 800 (S103). As a method for displaying the initial sound pressure distribution, which is represented as three-dimensional data, a slice image or maximum intensity projection (MIP) image along a certain section can be created and displayed. Any display method may be employed as long as the displayed image allows the user to designate the second region that the user wishes to check, such as a positon of a tumor.

Then, the user designates the second region through the input unit 900. The second region acquiring unit 252 stores data of the region designated through the input unit 900 as the second region data 222 (S104). Note that without performing step S104, the process waits for the user to complete designating the second region.

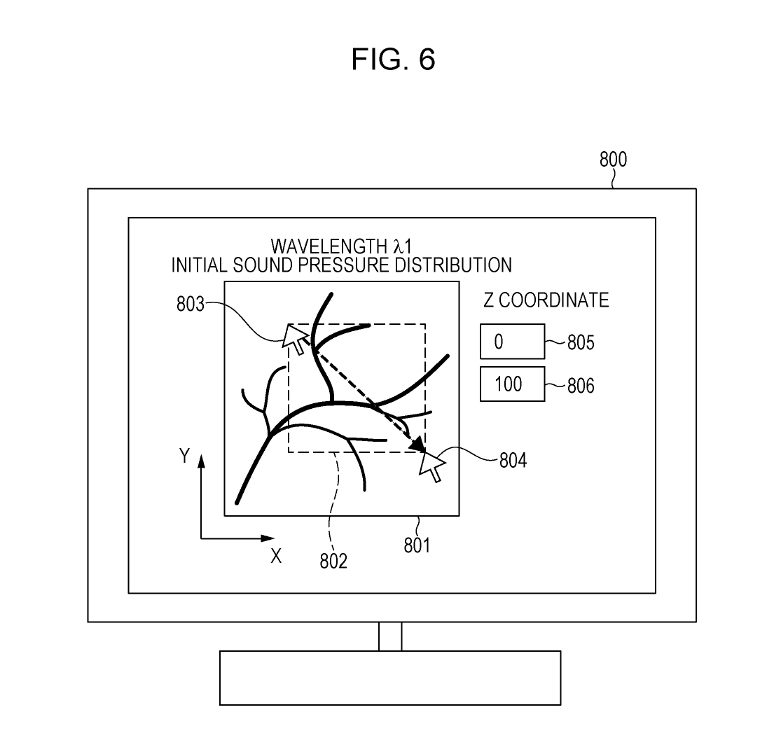

Here, an exemplary method for designating the second region will be described with reference to FIG. 6. In this exemplary method, the input unit 900 corresponds to a mouse and a keyboard. An MIP image of the initial sound pressure distribution along the Z direction is displayed in a display region 801 on the display unit 800. The user moves the mouse cursor to a start position 803 and starts a dragging operation. Then, the user moves the mouse cursor to an end position 804 and ends the dragging operation. Thus, a region 802 is defined. In addition, the user specifies a range in the Z direction in an item 805 and an item 806. A three-dimensional region is defined from the region 802 and the range in the Z direction and is set as the second region designated by the user. Note that the method for designating a region is not limited to this method.

Then, an optical absorption coefficient distribution is calculated (S105).

Here, the calculation of the optical absorption coefficient distribution will be described in detail with reference to FIG. 5D. The third region acquiring unit 253 determines a third region (S131). Then, the first light fluence distribution calculating unit 281 calculates a first light fluence distribution for a single pulse (S132). Then, the first optical absorption coefficient distribution calculating unit 311 calculates a first optical absorption coefficient distribution for a single pulse (S133) and causes the first optical absorption coefficient distribution data 321 to be stored in the storing unit 710 (S134). Here, the control unit 730 determines whether or not the calculation has been completed for the pulses used for irradiation in step S101 (S135). If the calculation has not been completed, the process returns to step S131; if the calculation has been completed, the calculation of the optical absorption coefficient distribution ends.

Then, the first optical absorption coefficient distribution calculating unit 311 averages the optical absorption coefficient distributions for a plurality of pulses, and the control unit 730 causes the averaged optical absorption coefficient distribution to be displayed on the display unit 800 (S106). As a method for displaying the optical absorption coefficient distribution, which is represented as three-dimensional data, a slice image or an MIP image along a section can be created and displayed.

Note that the optical absorption coefficient distribution may be calculated and displayed again by changing the third region. For example, first, an optical absorption coefficient distribution obtained by calculating a light fluence distribution in the third region having a small margin from the second region is displayed. Then, a third region having a larger margin from the second region than in the previous calculation may be newly set, and an optical absorption coefficient distribution obtained by calculating a light fluence distribution in the newly set third region may be displayed. In this case, upon completion of the calculation of the light fluence distribution in the previously set third region, the calculation of a light fluence distribution in the larger third region may be started.

According to the first embodiment, it is possible to shorten the time taken to calculate an optical absorption coefficient distribution by reducing the calculation region for a light fluence distribution on the basis of the second region designated by the user. Accordingly, it is possible to present the optical absorption coefficient distribution to the user in a shorter time.

It is also possible to reduce data sizes of the light fluence distribution data and the optical absorption coefficient distribution data, enabling the capacity of a storing unit to be reduced.

### Second Embodiment

A photoacoustic apparatus according to a second embodiment is an apparatus that repeats a plurality of irradiations with pulsed light beams at two different wavelengths (first wavelength λ1 and second wavelength λ2), unlike in the first embodiment using the same wavelength, and that displays an oxygen saturation degree distribution by reconstructing reception signals of an obtained photoacoustic wave.

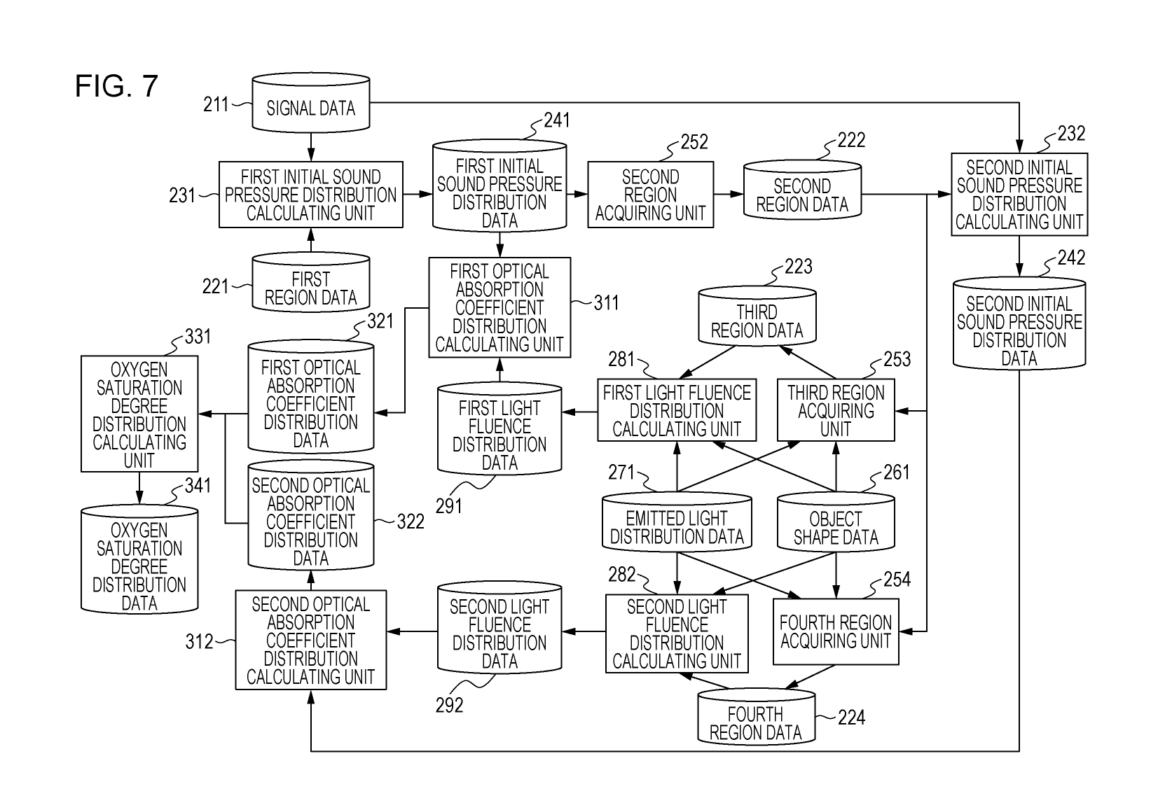

FIG. 7 illustrates a dataflow according to this embodiment. The above-described data and modules are denoted by the same reference numerals and omitted from description.

In this embodiment, signal data includes, in addition to data of an electric signal, which is a first signal, corresponding to the first wavelength λ1, data of an electric signal, which is a second signal, corresponding to the second wavelength λ2.

### Second Initial Sound Pressure Distribution Calculating Unit 232 and Second Initial Sound Pressure Distribution Data 242

A second initial sound pressure distribution calculating unit 232 receives the signal data 211 and the second region data 222, calculates a second initial sound pressure distribution corresponding to the second wavelength λ2 in the second region indicated by the second region data 222, and outputs data of the distribution as second initial sound pressure distribution data 242. The second initial sound pressure distribution calculating unit 232 may calculate the second initial sound pressure distribution by using a known reconstruction algorithm.

Unlike the first initial sound pressure distribution calculating unit 231 calculating the initial sound pressure distribution in the first region, the second initial sound pressure distribution calculating unit 232 calculates the initial sound pressure distribution in the second region desired by the user. It is sufficient for the second initial sound pressure distribution calculating unit 232 to calculate the initial sound pressure distribution in the second region that is smaller than the first region. Accordingly, the calculation amount is smaller than in the calculation performed by the first initial sound pressure distribution calculating unit 231.

### Fourth Region Acquiring Unit 254 and Fourth Region Data 224

In a manner similar to the third region acquiring unit 253, a fourth region acquiring unit 254 receives the second region data 222, the object shape data 261, and the emitted light distribution data 271 and outputs fourth region data 224. A fourth region indicated by the fourth region data 224 is to be set as a calculation region for a light fluence distribution corresponding to the second wavelength λ2, which will be described later.

The fourth region data 224 is data indicating the fourth region that includes at least the second region and an irradiated region of the object surface irradiated with the pulsed light 130. In addition, the fourth region data 224 is data indicating a region that is larger than the second region and smaller than the first region.

Note that the fourth region acquiring unit 254 may calculate the fourth region data 224 by reading the object shape data 261 and the emitted light distribution data 271 from the storing unit 710, the object shape data 261 and the emitted light distribution data 271 being obtained when the object 1000 is irradiated with the pulsed light 130 at the second wavelength λ2.

In addition, the fourth region data 224 and the third region data 223 may be identical to each other. In this case, the fourth region acquiring unit 254 may not newly calculate the fourth region data 224 and may read the third region data 223 from the storing unit 710 and may output the third region data 223 as the fourth region data 224.

### Second Light Fluence Distribution Calculating Unit 282 and Second Light Fluence Distribution Data 292

A second light fluence distribution calculating unit 282 receives the fourth region data 224, the object shape data 261, and the emitted light distribution data 271 and calculates propagation of light at the second wavelength λ2 used for irradiation of the object 1000 by setting the fourth region as a calculation region. As a result, the second light fluence distribution calculating unit 282 acquires a second light fluence distribution corresponding to the second wavelength λ2 in the fourth region and outputs data of the distribution as second light fluence distribution data 292. Since the fourth region is a region including the second region, the second region is also included in a calculation target by setting the fourth region as a calculation region. That is, the second light fluence distribution calculating unit 282 also calculates the light fluence distribution in the second region as a result of calculation of the light fluence distribution in the fourth region.

### Second Optical Absorption Coefficient Distribution Calculating Unit 312 and Second Optical Absorption Coefficient Distribution Data 322

A second optical absorption coefficient distribution calculating unit 312 receives the second region data 222, the second initial sound pressure distribution data 242, and the second light fluence distribution data 292. Then, the second optical absorption coefficient distribution calculating unit 312 calculates a second optical absorption coefficient distribution corresponding to the second wavelength λ2 in the second region 2002 and outputs data of the distribution as second optical absorption coefficient distribution data 322.

### Oxygen Saturation Degree Distribution Calculating Unit 331 and Oxygen Saturation Degree Distribution Data 341

An oxygen saturation degree distribution calculating unit 331 receives the second region data 222, the first optical absorption coefficient distribution data 321, and the second optical absorption coefficient distribution data 322, calculates an oxygen saturation degree distribution, and outputs data of the distribution as oxygen saturation degree distribution data 341. The oxygen saturation degree distribution calculating unit 331 serving as the object information calculating unit calculates the oxygen saturation degree distribution as the object information distribution.

In the case where main light absorbers in the object 1000 are oxyhemoglobin and deoxyhemoglobin, it is known that an oxygen saturation degree SO₂ as a constituent concentration is represented by the following Expression 5.

$${SO}_{2} = \frac{- \frac{{μ}_{a} ( {λ}_{1} )}{{μ}_{a} ( {λ}_{2} )} {ɛ}_{de} ( {λ}_{2} ) + {ɛ}_{de} ( {λ}_{1} )}{- \frac{{μ}_{a} ( {λ}_{1} )}{{μ}_{a} ( {λ}_{2} )} \{ {ɛ}_{de} ( {λ}_{2} ) + {ɛ}_{ox} ( {λ}_{2} ) \} + \{ {ɛ}_{de} ( {λ}_{1} ) + {ɛ}_{ox} ( {λ}_{1} ) \}} \qquad Expression 5$$

where μₐ(λ) is an optical absorption coefficient at a wavelength λ, and εₒₓ(λ) and ε_{de}(λ) are respectively the optical absorption coefficient of oxyhemoglobin and the optical absorption coefficient of deoxyhemoglobin each at the wavelength λ. Since εₒₓ(λ) and ε_{de}(λ) are known, the oxygen saturation degree distribution can be calculated from a plurality of optical absorption coefficient distributions corresponding to the respective wavelengths according to Expression 5.

The oxygen saturation degree distribution calculating unit 331 splits the region indicated by the second region data 222 into voxels having a predetermined size. Then, for each of the voxels, the oxygen saturation degree distribution calculating unit 331 calculates the oxygen saturation degree according to Expression 5 by using the first optical absorption coefficient corresponding to the first wavelength λ1 and the second optical absorption coefficient corresponding to the second wavelength λ2. The oxygen saturation degree distribution calculating unit 331 outputs data of the calculated oxygen saturation degree in each of the voxels as the oxygen saturation degree distribution data 341.

Note that although this embodiment has described the example of calculating the oxygen saturation degree distribution from the plurality of optical absorption coefficient distributions corresponding to different wavelengths, the calculated information is not limited to the oxygen saturation degree. The arithmetic unit 720 may calculate a concentration distribution of a substance constituting the object 1000 on the basis of the plurality of optical absorption coefficient distributions corresponding to different wavelengths. Examples of the concentration distribution of a substance include concentration distributions of water, fat, collagen, oxyhemoglobin, deoxyhemoglobin, and all types of hemoglobin.

### Image Display Method

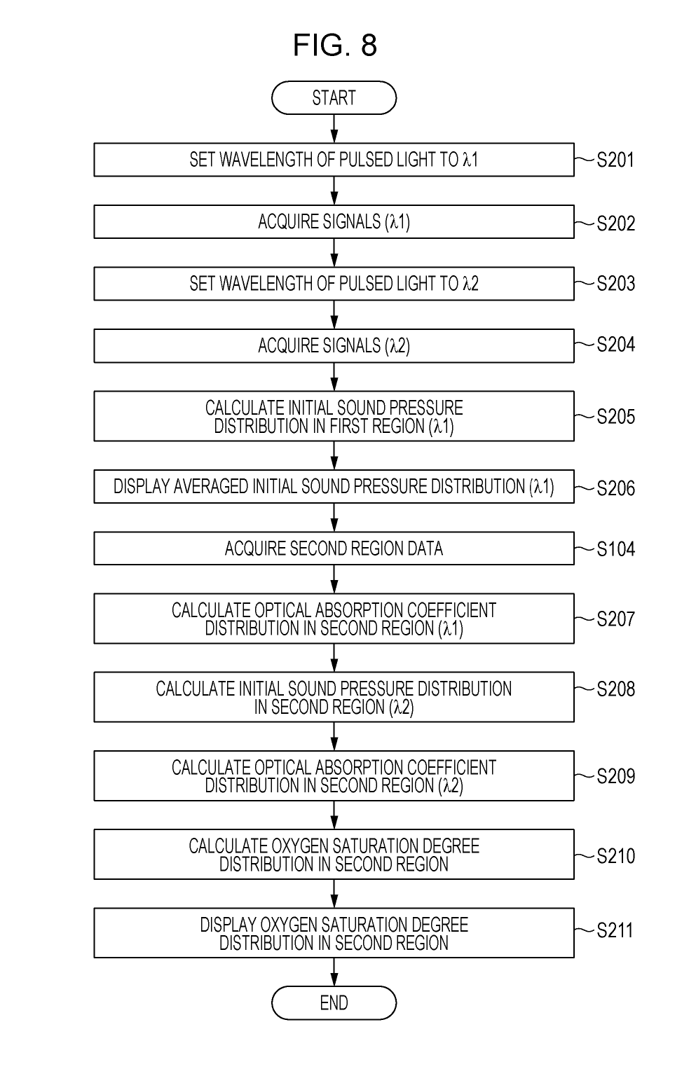

A method for displaying an image of the oxygen saturation degree distribution by using the photoacoustic apparatus according to this embodiment will be described with reference to FIG. 8.

First, the control unit 730 sets the wavelength of pulsed light generated by the light emitting unit 100 to the first wavelength λ1 (S201) and acquires signals by using the pulsed light at the wavelength λ1 (S202). The processing here is substantially the same as that in step S101.

Then, the control unit 730 sets the wavelength of pulsed light generated by the light emitting unit 100 to the second wavelength λ2 (S203) and acquires signals by using the pulsed light at the wavelength λ2 (S204). The processing here is substantially the same as that in step S101.

Then, by using the signal data acquired by using the pulsed light at the wavelength λ1, the first initial sound pressure distribution calculating unit 231 calculates a first initial sound pressure distribution (S205). The processing here is substantially the same as that in step S102.

Then, initial sound pressure distributions based on the pulsed light at the wavelength λ1 are averaged, and the averaged initial sound pressure distribution is displayed (S206). The processing here is substantially the same as that in step S103.

Then, second region data is acquired and stored (S104).

Then, the first optical absorption coefficient distribution calculating unit 311 calculates a first optical absorption coefficient distribution from the initial sound pressure distribution based on the pulsed light at the wavelength λ1 (S207). The processing here is substantially the same as that in step S105.

Then, by using the signals acquired by using the pulsed light at the wavelength λ2, the second initial sound pressure distribution calculating unit 232 calculates a second initial sound pressure distribution (S208). The processing here is substantially the same as that in step S102. Note that since the second region data 222 indicating a second region designated by the user in step S104 is stored in the storing unit 710, it is sufficient to perform calculation for a region that is smaller than the calculation region used in step S205.

Then, the second optical absorption coefficient distribution calculating unit 312 calculates a second optical absorption coefficient distribution from the initial sound pressure distribution based on the pulsed light at the wavelength λ2 (S209). The processing here is substantially the same as that in step S105.

Then, from the first and second optical absorption coefficient distributions based on the pulsed light at the wavelengths λ1 and λ2, the oxygen saturation degree distribution calculating unit 331 calculates an oxygen saturation degree distribution (S210).

Then, the control unit 730 causes the oxygen saturation degree distribution to be displayed on the display unit 800 (S211). The oxygen saturation degree distribution may be displayed with a portion masked, the portion whose value of a corresponding optical absorption coefficient distribution is in a predetermined range. As a display method, another known masking technique can be used.

Note that step S204 may be performed after step S104 so that signals are selectively acquired by using the pulsed light at the wavelength λ2 for the second region designated by the user. That is, in irradiation with each pulsed light beam, the movement range of the light emitting unit 100 may be changed so that the second region designated by the user is easily irradiated with the pulsed light. In addition, the number of pulses for irradiation may be changed.

According to the second embodiment, it is possible to reduce the calculation regions for the first light fluence distribution corresponding to the wavelength λ1, the second initial sound pressure distribution corresponding to the wavelength λ2, and the second light fluence distribution corresponding to the wavelength λ2 and to display the oxygen saturation degree distribution quickly. It is also possible to reduce the data sizes of the first light fluence distribution data corresponding to the wavelength λ1 and the first optical absorption coefficient distribution data corresponding to the wavelength λ1, enabling the capacity of the storing unit to be reduced. It is also possible to reduce the data sizes of the second initial sound pressure distribution data corresponding to the wavelength λ2, the second light fluence distribution data corresponding to the wavelength λ2, the second optical absorption coefficient distribution data corresponding to the wavelength λ2, and the oxygen saturation degree data, enabling the capacity of the storing unit to be reduced.

### Third Embodiment

A photoacoustic apparatus according to a third embodiment is a system that sequentially displays an initial sound pressure distribution and an optical absorption coefficient distribution by reconstructing reception signals of an obtained photoacoustic wave. In addition, the photoacoustic apparatus has a function of, at the time the user designates a region, limiting a calculation region for information to be acquired later.

The system configuration and dataflow are substantially the same as those in the first embodiment.

### Image Display Method

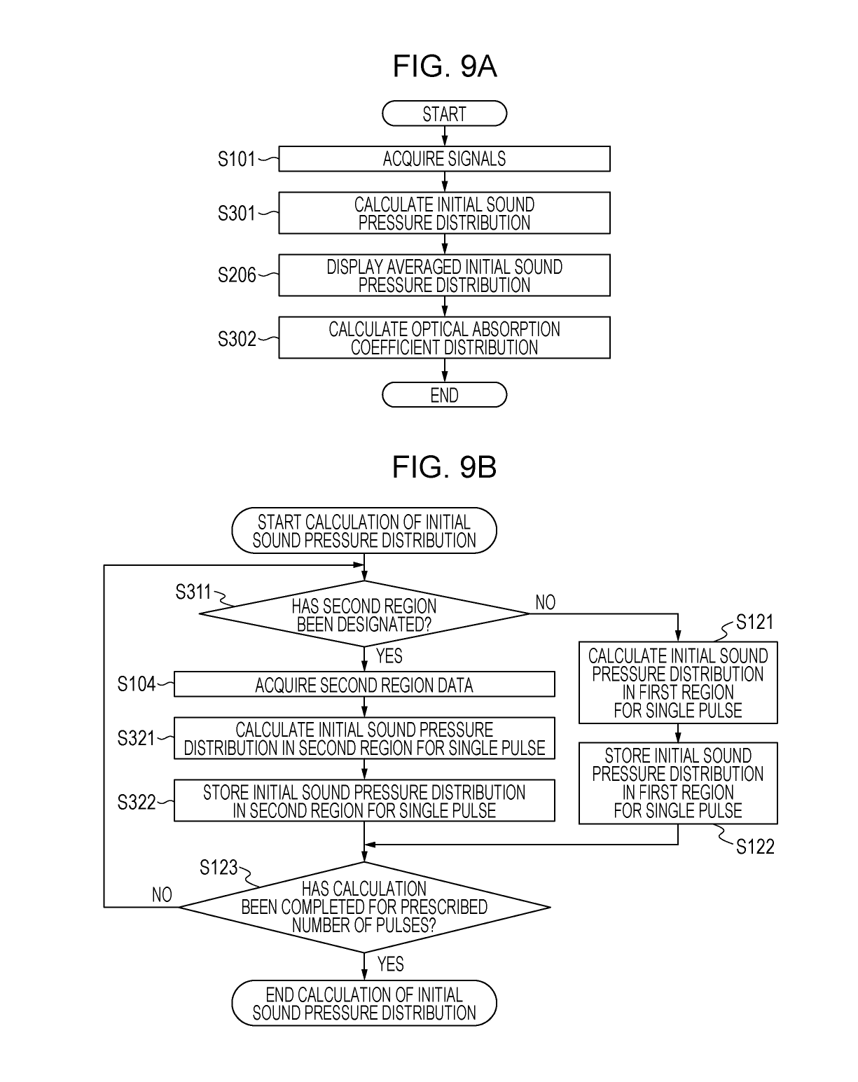

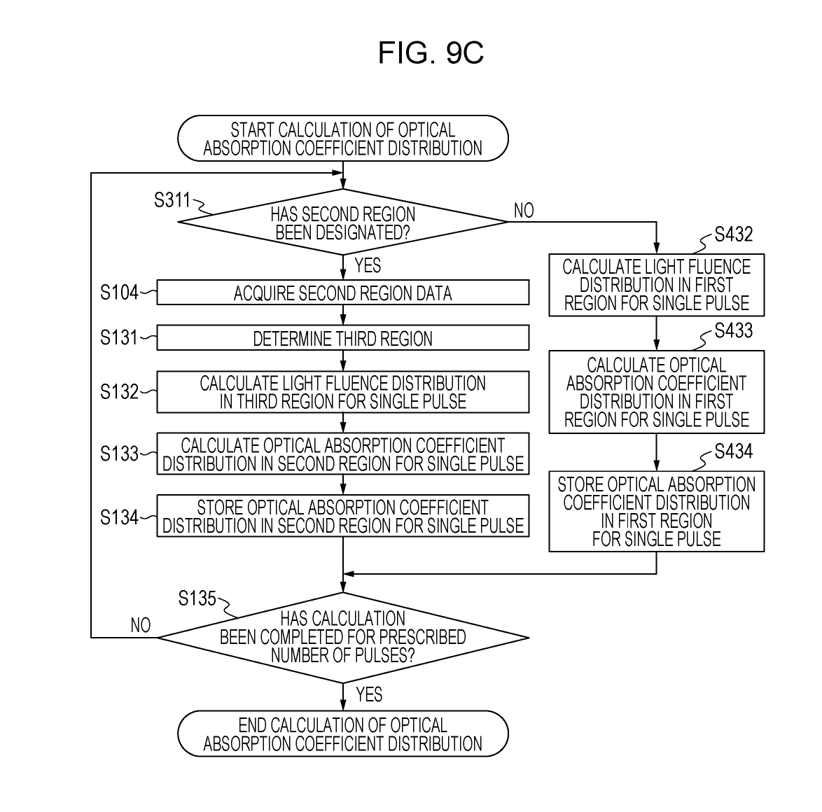

A method for displaying an image of an optical absorption coefficient distribution by using the photoacoustic apparatus according to this embodiment will be described with reference to FIGS. 9A to 9C. FIG. 9A is an overall flowchart, and FIGS. 9B and 9C are flowcharts respectively illustrating details of initial sound pressure distribution calculation and optical absorption coefficient distribution calculation according to the third embodiment. The flowcharts are different from those in the second embodiment in that a region can be designated at an arbitrary timing during the initial sound pressure distribution calculation or the optical absorption coefficient distribution calculation.

First, signals are acquired (S101).

Then, an initial sound pressure distribution is calculated by using data of the signals (S301).

Here, the calculation of the initial sound pressure distribution will be described in detail with reference to FIG. 9B. First, the control unit 730 determines whether or not a second region has been designated (S311). For example, this determination can be performed by the following method. A flag indicating whether or not the second region has been newly designated is prepared and is initialized to 0 at the time of start-up. In addition, at the time the control unit 730 is notified of the designation of the second region from the input unit 900, the flag is changed to 1. Upon the change of the flag to 1, the control unit 730 determines in step S311 that the second region has been designated. If it is determined that the second region has been designated, the second region acquiring unit 252 stores data of the designated second region as the second region data 222 (S104), and the process proceeds to step S321. Then, an initial sound pressure distribution in the second region for a single pulse is calculated (S321), and data thereof is stored (S322).

On the other hand, if it is not determined that the second region has been designated, step S104 is not performed, and the process proceeds to step S121. Then, an initial sound pressure distribution in a first region for a single pulse is calculated (S121), and data thereof is stored (S122). Finally, it is determined whether or not the calculation has been completed for a prescribed number of pulses (S123). If the calculation has been completed, the calculation of the initial sound pressure distribution ends; if not, the process returns to step S311.

Then, the averaged initial sound pressure distribution based on the wavelength λ1 is displayed on the display unit 800 (S206). If the second region is designated prior to step S206, among calculated initial sound pressure distributions for a plurality of pulses, only the initial sound pressure distribution in the second region may be averaged.

Finally, it is determined whether or not the calculation has been completed for a prescribed number of pulses (S123). If the calculation has been completed, the calculation of the initial sound pressure distribution ends; if not, the process returns to step S311.

In the first embodiment, at the time the initial sound pressure distribution is displayed, the process waits for the user to designate the second region. However, in this embodiment, the process is continued without waiting.

Then, from the initial sound pressure distribution based on the pulsed light at the wavelength λ1, an optical absorption coefficient distribution is calculated (S302).

Here, the calculation of the optical absorption coefficient distribution will be described in detail with reference to FIG. 9C.

First, the control unit 730 determines whether or not the second region has been designated (S311). If it is determined that the second region has been designated, data of the designated second region is stored as the second region data 222 (S104), and the process proceeds to step S131. Then, the third region acquiring unit 253 determines a third region (S131). In addition, a light fluence distribution for a single pulse is calculated by setting the third region as a calculation region (S132), and an optical absorption coefficient distribution in the second region for a single pulse is calculated (S133), and data thereof is stored (S134).

On the other hand, if it is not determined that the second region has been newly designated, step S104 is not performed, and the process proceeds to step S432. Then, a light fluence distribution in the first region is calculated by setting the first region as a calculation region (S432). Then, by using the initial sound pressure distribution in the first region and the light fluence distribution in the first region, an optical absorption coefficient distribution in the first region is calculated (S433), and data thereof is stored (S434).

Finally, it is determined whether or not the calculation has been completed for a prescribed number of pulses (S135). If the calculation has been completed, the calculation of the optical absorption coefficient distribution ends; if not, the process returns to step S311.

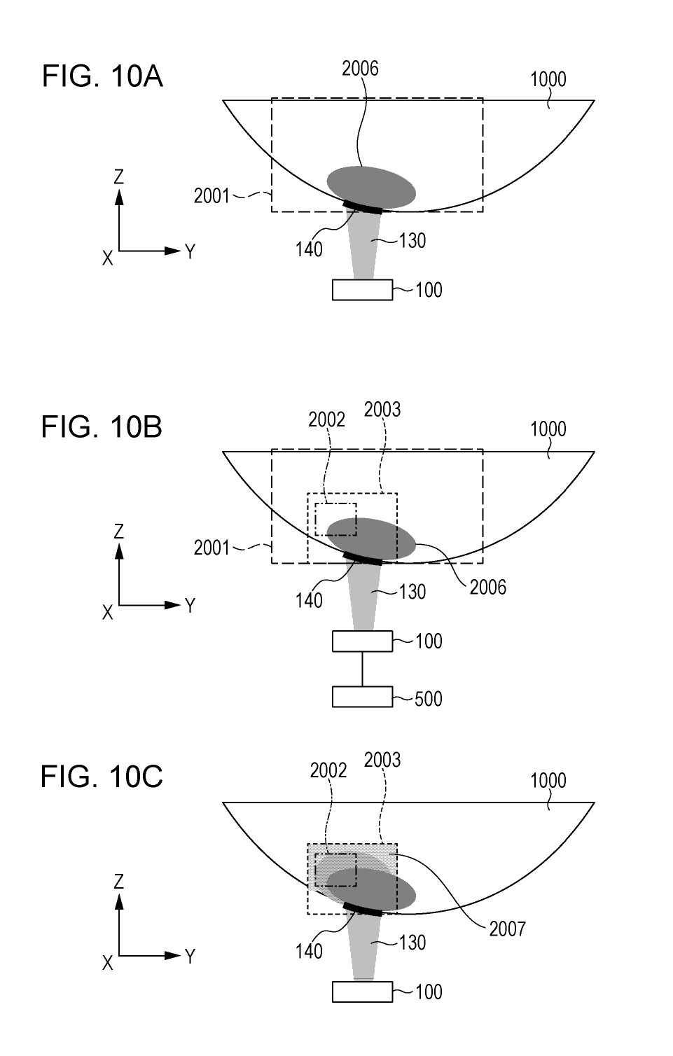

Note that in the case where the second region is designated during the calculation of propagation of light by setting the first region as a calculation region in step S432, the light fluence distribution calculated up to this point may be used for calculation later. That is, on the basis of the light fluence distribution obtained by setting the first region as a calculation region, the light fluence distribution in the third region may be calculated by calculating propagation of light by setting the third region as a calculation region. An example of this case will be described with reference to FIGS. 10A to 10C.

FIG. 10A illustrates a process in step S432 for calculation of propagation of the pulsed light 130 by setting the first region 2001 as a calculation region, the pulsed light 130 being emitted onto the irradiated region 140. A light fluence distribution 2006 represents a light fluence distribution of the pulsed light 130 calculated up to this time point.

FIG. 10B illustrates the third region 2003 determined in step S131 in the case where the second region 2002 has been designated by the user through the input unit 900. Then, although the first light fluence distribution calculating unit 281 is to calculate propagation of light of the pulsed light 130 by setting the third region 2003 as a calculation region, the light fluence distribution 2006 obtained at this time point by setting the first region 2001 as a calculation region is calculated. Accordingly, the first light fluence distribution calculating unit 281 may calculate the propagation of light by setting the third region 2003 as a calculation region and by using the light fluence distribution 2006 as a source.

FIG. 10C illustrates a light fluence distribution 2007 calculated from the calculation of propagation of light by setting the third region 2003 as a calculation region and by using the light fluence distribution 2006 as a source.

In this method, it is possible to shorten the time taken to complete the calculation of the light fluence distribution 2007. In addition, since the light fluence distribution 2006 is a light fluence distribution obtained by setting, as a calculation region, the first region 2001 that is larger than the third region 2003, the quantitativeness is high. Thus, in some cases, the quantitativeness may be higher in the light fluence distribution 2007 obtained on the basis of the light fluence distribution 2006 than in the light fluence distribution obtained by setting the third region 2003 as a calculation region from the start.

According to the third embodiment, even if time passes from the display of the initial sound pressure distribution to the designation of the second region by the user, calculation for the first region can proceed in the background. Accordingly, it is possible to present object information in the region designated by the user more quickly than in the first embodiment.

Note that in the case where measurement is performed by using light beams at a plurality of wavelengths, calculation may proceed in the background while the user does not designate a region.

### OTHER EMBODIMENTS

Embodiments or certain aspects the embodiments of the present invention can be implemented by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiments, and by a method performed by the computer of the system or apparatus by, for example, reading out and executing the computer executable instructions from the storage medium to perform the functions of one or more of the above-described embodiments and/or controlling the one or more circuits to perform the functions of one or more of the above-described embodiments. The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all modifications and equivalent structures and functions.

This application claims the benefit of Japanese Patent Application No. 2015-187464, filed Sep. 24, 2015, which is hereby incorporated by reference herein in its entirety.

What is claimed is:

1. A photoacoustic apparatus comprising:
a light emitting unit that emits light onto an object;
a receiving unit that receives a photoacoustic wave generated by irradiation of the object with the light and outputs a signal;
a first calculating unit that calculates an initial sound pressure distribution in a first region on the basis of the signal;
a control unit that causes the initial sound pressure distribution to be displayed on a display unit;
a region acquiring unit that acquires information on a second region designated within a region of the initial sound pressure distribution displayed on the display unit, the second region being smaller than the first region, and information on a third region on the basis of the information on the second region, the third region being larger than the second region and smaller than the first region;
a second calculating unit that calculates, on the basis of the information on the third region, propagation of the light emitted onto the object by setting the third region as a calculation region to calculate a light fluence distribution in the second region; and
a third calculating unit that calculates, on the basis of the initial sound pressure distribution and the light fluence distribution, an object information distribution in the second region.

2. A photoacoustic apparatus comprising:
a light emitting unit that emits light onto an object;
a receiving unit that receives a photoacoustic wave generated by irradiation of the object with the light from the light emitting unit to output a signal;
a first calculating unit that calculates an initial sound pressure distribution in a first region on the basis of the signal;
a control unit that causes the initial sound pressure distribution to be displayed on a display unit;
a region acquiring unit that acquires information on a second region designated within a region of the initial sound pressure distribution displayed on the display unit, the second region being smaller than the first region;
a second calculating unit that calculates, on the basis of the information on the second region, propagation of the light emitted onto the object by setting a third region as a calculation region to calculate a light fluence distribution in the second region, the third region including the second region and an irradiated region on a surface of the object irradiated with the light and being smaller than the first region; and
a third calculating unit that calculates, on the basis of the initial sound pressure distribution and the light fluence distribution, an object information distribution in the second region.

3. The photoacoustic apparatus according to claim 1, further comprising an input unit configured to enable the first region or the second region to be designated.

4. The photoacoustic apparatus according to claim 1, wherein
until the second region is designated, the second calculating unit calculates propagation of the light by setting the first region as the calculation region, and
once the second region is designated, the second calculating unit calculates, on the basis of a light fluence distribution acquired by setting the first region as the calculation region, propagation of the light by setting the third region as the calculation region to calculate the light fluence distribution in the second region.

5. The photoacoustic apparatus according to claim 1, wherein the region acquiring unit acquires the information on the third region having a margin from the second region in an out-of-plane direction of an irradiated region of the light used for irradiation of a surface of the object, the margin being smaller than a margin from the second region in an in-plane direction thereof.

6. The photoacoustic apparatus according to claim 2, wherein
until the second region is designated, the second calculating unit calculates propagation of the light by setting the first region as the calculation region, and
once the second region is designated, the second calculating unit calculates, on the basis of a light fluence distribution acquired by setting the first region as the calculation region, propagation of light by setting the third region as the calculation region to calculate the light fluence distribution in the second region.

7. The photoacoustic apparatus according to claim 2, wherein the region acquiring unit acquires information on the third region having a margin from the second region in an out-of-plane direction of an irradiated region of the light used for irradiation of a surface of the object, the margin being smaller than a margin from the second region in an in-plane direction thereof.

8. The photoacoustic apparatus according to claim 2, wherein the control unit causes the object information distribution in the second region to be displayed on the display unit.

9. The photoacoustic apparatus according to claim 1, wherein the third calculating unit calculates an optical absorption coefficient distribution as the object information distribution.

10. The photoacoustic apparatus according to claim 1, wherein the control unit causes the object information distribution in the second region to be displayed on the display unit.

11. A photoacoustic apparatus comprising:
a light emitting unit that emits light onto an object;
a receiving unit that receives a photoacoustic wave generated by irradiation of the object with the light from the light emitting unit to output a signal;
a first calculating unit that calculates an initial sound pressure distribution in a first region on the basis of the signal;
a second calculating unit that calculates propagation of the light emitted onto the object by setting a region larger than the first region as a calculation region to calculate a light fluence distribution in the first region; and
a third calculating unit that calculates, on the basis of the initial sound pressure distribution and the light fluence distribution, an object information distribution in the first region.

12. The photoacoustic apparatus according to claim 11, further comprising an input unit configured to enable the first region to be designated.

13. The photoacoustic apparatus according to claim 11, wherein the second calculating unit calculates the light fluence distribution in the first region by setting, as the calculation region, a region having a margin from the first region in an out-of-plane direction of an irradiated region of the light used for irradiation of a surface of the object, the margin being smaller than a margin from the second region in an in-plane direction thereof.

14. The photoacoustic apparatus according to claim 11, further comprising a control unit that causes the object information distribution in the first region to be displayed on a display unit.

15. A photoacoustic apparatus comprising:
a light emitting unit that emits light onto an object;
a receiving unit that receives a photoacoustic wave generated by irradiation of the object with the light from the light emitting unit to output an electric signal;
a first calculating unit that calculates an initial sound pressure distribution;
a second calculating unit that calculates a light fluence distribution;
a third calculating unit that calculates an object information distribution,
the light emitting unit emitting the light at a first wavelength onto the object,
the receiving unit receiving the photoacoustic wave corresponding to the first wavelength, the photoacoustic wave being generated by the irradiation of the object with the light at the first wavelength, to output a first signal corresponding to the first wavelength,
the light emitting unit emitting the light at a second wavelength onto the object,
the receiving unit receiving the photoacoustic wave corresponding to the second wavelength, the photoacoustic wave being generated by the irradiation of the object with the light at the second wavelength, to output a second signal corresponding to the second wavelength,
the first calculating unit calculating, on the basis of the first signal, a first initial sound pressure distribution in a first region corresponding to the first wavelength;
a control unit that causes the first initial sound pressure distribution to be displayed on a display unit; and
a region acquiring unit that acquires information on a second region designated within a region of the first initial sound pressure distribution displayed on the display unit, the second region being smaller than the first region, and on the basis of the information on the second region, acquires information on a third region and a fourth region that are each larger than the second region and smaller than the first region, wherein
the second calculating unit calculates, on the basis of the information on the third region, propagation of the light at the first wavelength emitted onto the object by setting the third region as a calculation region to calculate a first light fluence distribution corresponding to the first wavelength in the second region,
the first calculating unit calculates, on the basis of the second signal, a second light fluence distribution corresponding to the second wavelength in the second region,
the second calculating unit calculates, on the basis of the information on the fourth region, propagation of the light at the second wavelength emitted onto the object by setting the fourth region as a calculation region to calculate a second light fluence distribution corresponding to the second wavelength in the second region, and
the third calculating unit calculates an object information distribution in the second region on the basis of the first initial sound pressure distribution, the second initial sound pressure distribution, the first light fluence distribution, and the second light fluence distribution.

16. The photoacoustic apparatus according to claim 15, wherein the third calculating unit calculates a substance concentration distribution as the object information distribution.

17. The photoacoustic apparatus according to claim 15, wherein the control unit causes the object information distribution in the second region to be displayed on the display unit.

18. A method comprising:
setting a first region;
displaying an initial sound pressure distribution in the first region;
setting a second region that is smaller than the first region;
setting a third region that is larger than the second region and smaller than the first region;
calculating propagation of light emitted onto an object by setting the third region as a calculation region to calculate a light fluence distribution in the second region; and
calculating an object information distribution in the second region on the basis of the initial sound pressure distribution and the light fluence distribution.

19. The method according to claim 18, further comprising displaying the object information distribution in the second region.

20. A non-transitory storage medium having a program stored therein, the program causing a computer to execute the method according to claim 18.
